(19) United States
(12) Patent Application Publication
(10) Pub. No.: US 2007/0218104 A1
(43) Pub. Date: Sep. 20, 2007

(54) Rate controlled release of a pharmaceutical agent in a biodegradable device

(75) Inventors: Jay Kunzler, Canandaigua, NY (US); Joseph Salamone, Boca Raton, FL (US)

Correspondence Address: Bausch & Lomb Incorporated, One Bausch & Lomb Place, Rochester, NY, 14604-2701, US

(73) Assignee: Bausch & Lomb Incorporation

(21) Appl. No.: 11/376,080

(22) Filed: Mar. 15, 2006

(51) Int. Cl. A61K 9/14; A61K 31/557

(52) U.S. Cl. 424/427; 424/487; 514/573

(57) ABSTRACT

Matrix controlled diffusion drug delivery systems comprising a therapeutically effective amount of one or more pharmaceutically active agents polymerized with a monomeric mixture comprising one or more acrylate ester and/or methacrylate ester-containing monomers and one or more acrylamido-containing monomers are provided. Processes for their preparation and methods of use are also disclosed.

### BACKGROUND OF THE INVENTION

1. Technical Field

The present invention relates generally to hydrogels, drug delivery systems, and methods of treatment.

2. Description of Related Art

Conventional drug delivery involving frequent periodic dosing is not ideal or practical in many instances. For example, with more toxic drugs, conventional periodic dosing can result in high initial drug levels at the time of dosing, followed by low drug levels between doses often times below levels of therapeutic value. Likewise, conventional periodic dosing may not be practical or therapeutically effective in certain instances such as with pharmaceutical therapies targeting areas of the inner eye or brain in need of treatment such as the retina.

During the last two decades, significant advances have been made in the design of controlled release drug delivery systems. See, e.g., U.S. Patent Application Publication Nos. 2004/0043067 and 2004/0253293. Such advances have been made in an attempt to overcome some of the drug delivery shortcomings noted above. In general, controlled release drug delivery systems include both sustained drug delivery systems designed to deliver a drug for a predetermined period of time, and targeted drug delivery systems designed to deliver a drug to a specific area or organ of the body. Sustained and/or targeted controlled release drug delivery systems may vary considerably by mode of drug release within three basic drug controlled release categories. Basic drug controlled release categories include diffusion controlled release, chemical erosion controlled release and solvent activation controlled release. In a diffusion controlled release drug delivery system, a drug is surrounded by an inert barrier and diffuses from an inner reservoir, or a drug is dispersed throughout a polymer and diffuses from the polymer matrix. In a chemical erosion controlled release drug delivery system, a drug is uniformly distributed throughout a biodegradable polymer. The biodegradable polymer is designed to degrade as a result of hydrolysis to then uniformly release the drug. In a solvent activation controlled release drug delivery system, a drug is immobilized on polymers within a drug delivery system. Upon solvent activation, the solvent sensitive polymer degrades or swells to release the drug. Unfortunately, controlled release drug delivery systems to date do not provide a means by which one may manipulate and control drug delivery systems' drug release rate for specific drugs over a broad range of drugs.

Because of the noted shortcomings of current controlled release drug delivery systems, a need exists for controlled release drug delivery systems that allow for manipulation and control of drug release rates depending on the drug to be delivered, the location of delivery, the purpose of delivery and/or the therapeutic requirements of the individual patient.

### SUMMARY OF THE INVENTION

In accordance with one embodiment of the present invention, a matrix controlled diffusion drug delivery system is provided comprising a therapeutically effective amount of one or more pharmaceutically active agents polymerized with a monomeric mixture comprising one or more acrylate ester and/or methacrylate ester-containing monomers and one or more acrylamido-containing monomers.

In accordance with a second embodiment of the present invention, a matrix controlled diffusion drug delivery system is provided comprising a therapeutically effective amount of one or more pharmaceutically active agents polymerized with a copolymer obtained from the copolymerization of one or more acrylate ester and/or methacrylate ester-containing monomers with one or more acrylamido-containing monomers.

In accordance with a third embodiment of the present invention, a matrix controlled diffusion drug delivery system is provided comprising a therapeutically effective amount of one or more pharmaceutically active agents polymerized with a copolymer comprising one or more acrylate ester and/or methacrylate ester-containing units and one or more acrylamido-containing units.

In accordance with a fourth embodiment of the present invention, a process for preparing a matrix controlled diffusion drug delivery system is provided, the process comprising polymerizing a therapeutically effective amount of one or more pharmaceutically active agents with a monomeric mixture comprising one or more acrylate ester and/or methacrylate ester-containing monomers and one or more acrylamido-containing monomers.

In accordance with a fifth embodiment of the present invention, a process for preparing a matrix controlled diffusion drug delivery system is provided, the process comprising polymerizing a therapeutically effective amount of one or more pharmaceutically active agents with a copolymer obtained from the copolymerization of one or more acrylate ester and/or methacrylate ester-containing monomers with one or more acrylamido-containing monomers.

In accordance with a sixth embodiment of the present invention, a process for preparing a matrix controlled diffusion drug delivery system is provided, the process comprising polymerizing a therapeutically effective amount of one or more pharmaceutically active agents with a copolymer obtained from the copolymerization of one or more acrylate ester and/or methacrylate ester-containing monomers with one or more acrylamido-containing monomers.

In accordance with a seventh embodiment of the present invention, a process for preparing a matrix controlled diffusion drug delivery system is provided, the process comprising polymerizing a therapeutically effective amount of one or more pharmaceutically active agents with a copolymer comprising one or more acrylate ester and/or methacrylate ester-containing units and one or more acrylamido-containing units.

In accordance with an eighth embodiment of the present invention, a method of treating an ophthalmic state, disease, disorder, injury or condition is provided, the method comprising administering to a mammal in need of such treatment a matrix controlled diffusion drug delivery system comprising a therapeutically effective amount of one or more pharmaceutically active agents polymerized with a monomeric mixture comprising one or more acrylate ester and/or methacrylate ester-containing monomers and one or more acrylamido-containing monomers.

In accordance with a ninth embodiment of the present invention, a method of treating an ophthalmic state, disease, disorder, injury or condition is provided, the method comprising administering to a mammal in need of such treatment a matrix controlled diffusion drug delivery system comprising a therapeutically effective amount of one or more pharmaceutically active agents polymerized with a copolymer obtained from the copolymerization of one or more acrylate ester and/or methacrylate ester-containing monomers with one or more acrylamido-containing monomers.

In accordance with a tenth embodiment of the present invention, a method of treating an ophthalmic state, disease, disorder, injury or condition is provided, the method comprising administering to a mammal in need of such treatment a matrix controlled diffusion drug delivery system comprising a therapeutically effective amount of one or more pharmaceutically active agents polymerized with a copolymer comprising one or more acrylate ester and/or methacrylate ester-containing units and one or more acrylamido-containing units.

By polymerizing a therapeutically effective amount of one or more pharmaceutically active agents with a monomeric mixture including at least one or more acrylate ester or methacrylate ester-containing monomers and one or more acrylamido-containing monomers and copolymers thereof, a matrix controlled diffusion drug delivery system can advantageously be designed to allow for manipulation and control of drug release rates such that treatment of a disease, disorder, injury or condition in a mammal may be achieved.

The term “monomer” and like terms as used herein denote relatively low molecular weight compounds that are polymerizable by, for example, free radical polymerization, as well as higher molecular weight compounds also referred to as “prepolymers”, “macromonomers”, and related terms.

The term “treating” or “treatment” of a state, disease, disorder, injury or condition as used herein shall be understood to mean (1) preventing or delaying the appearance of clinical symptoms of the state, disease, disorder, injury or condition developing in a mammal that may be afflicted with or predisposed to the state, disease, disorder, injury or condition but does not yet experience or display clinical or subclinical symptoms of the state, disease, disorder, injury or condition, (2) inhibiting the state, disease, disorder, injury or condition, i.e., arresting or reducing the development of the disease or at least one clinical or subclinical symptom thereof, or (3) relieving the state, disease, disorder, injury or condition, i.e., causing regression of the state, disease, disorder, injury or condition or at least one of its clinical or subclinical symptoms.

The term “delivering” as used herein shall be understood to mean providing a therapeutically effective amount of a pharmaceutically active agent to a particular location within a host causing a therapeutically effective concentration of the pharmaceutically active agent at the particular location.

The term “subject” or “patient” or “host” or “mammal” as used herein refers to mammalian animals and humans.

### BRIEF DESCRIPTION OF THE DRAWINGS

FIG. 1 is a graphical representation depicting the percent cumulative drug release rate over time for a N,N-dimethylacrylamide (DMA)/methylmethacrylate (MMA) copolymer loaded with 20% w/w fluocinolone acetonide.

FIG. 2 is a graphical representation depicting the percent cumulative drug release rate over time for a DMA/MMA copolymer loaded with 40% w/w fluocinolone acetonide.

### DETAILED DESCRIPTION OF THE PREFERRED EMBODIMENTS

The present invention is directed to matrix controlled diffusion drug delivery systems of the present invention for the treatment of a state, disease, disorder, injury or condition in a mammal in need of treatment such as an ophthalmic disease in a mammal. The drug delivery systems will include at least a therapeutically effective amount of one or more pharmaceutically active agents polymerized with a monomeric mixture including at least one or more acrylate ester and/or methacrylate ester-containing monomers and one or more acrylamido-containing monomers and copolymers thereof.

The rate of release of the pharmaceutically active agents can be controlled by manipulating the hydrophobic/hydrophilic balance of the one or more acrylate ester and/or methacrylate ester-containing monomers and one or more acrylamido-containing monomers to achieve the desired rate of drug release, e.g., by copolymerizing a monomeric mixture containing at least one or more acrylate ester and/or methacrylate ester-containing monomers and one or more acrylamido-containing monomers in the presence of the pharmaceutically active agent(s), properties such as, for example, water content, modulus and glass transition temperature (T_{g}), can be controlled thereby having a pronounced impact on the release characteristics. For example, in the case of fluocinolone acetonide, a relatively hydrophobic drug, it is believed that the release rate can be changed significantly with respect to the water content of the system. Accordingly, the desired rate of drug release may be determined based on, for example, the drug to be delivered, the location of delivery, the copolymer used in making the drug delivery system, the purpose of delivery and/or the therapeutic requirements of the individual patient.

Suitable acrylate ester and/or methacrylate ester-containing monomers may be represented by the general formula: 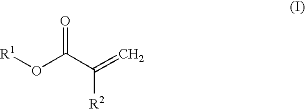
wherein R¹ may be a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, ether or polyether containing groups, substituted or unsubstituted, linear or branched, and R² is H or CH₃.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl (isopropyl), n-butyl, n-pentyl, etc., and the like.

Representative examples of cycloalkyl groups for use herein include, by way of example, a substituted or unsubstituted non-aromatic mono or multicyclic ring system of about 3 to about 18 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 18 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 18 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 25 carbon atoms such as, for example, phenyl, naphthyl, tetrahydronapthyl, indenyl, biphenyl and the like, optionally containing one or more heteroatoms, e.g., O and N, and the like.

Representative examples of arylalkyl groups for use herein include, by way of example, a substituted or unsubstituted aryl group as defined above directly bonded to an alkyl group as defined above, e.g., —CH₂C₆H₅, —C₂H₅C₆H₅ and the like, wherein the aryl group can optionally contain one or more heteroatoms, e.g., O and N, and the like.

Representative examples of ether or polyether containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are defined above, e.g., alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures or copolymers thereof, an ether or polyether group of the general formula —R³OR⁴, wherein R³ is a bond, an alkyl, cycloalkyl or aryl group as defined above and R⁴ is an alkyl, cycloalkyl or aryl group as defined above, e.g., —CH₂CH₂OC₆H₅ and —CH₂CH₂OC₂H₅, and the like.

The substituents in the ‘substituted alkyl’, ‘substituted cycloalkyl’, ‘substituted cycloalkylalkyl’, ‘substituted cycloalkenyl’, ‘substituted arylalkyl’ and ‘substituted aryl’ may be the same or different with one or more selected from the group such as hydrogen, halogen (e.g., fluorine), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted aryl, substituted or unsubstituted heteroaryl, substituted heterocyclylalkyl ring, substituted or unsubstituted heteroarylalkyl, substituted or unsubstituted heterocyclic ring.

In one embodiment, useful acrylate ester or methacrylate ester-containing monomers include, but are not limited to, a linear or branched, substituted or unsubstituted, C₁ to C₁₈ alkyl acrylate, a linear or branched, substituted or unsubstituted, C₁ to C₁₈ alkyl methacrylate, a substituted or unsubstituted C₃ to C₁₈ cycloalkyl acrylate, a substituted or unsubstituted C₃ to C₁₈ cycloalkyl methacrylate, a substituted or unsubstituted C₆ to C₂₅ aryl or alkaryl acrylate, a substituted or unsubstituted C₆ to C₂₅ aryl or alkaryl methacrylate, an ethoxylated acrylate, an ethoxylated methacrylate, partially fluorinated acrylates, partially fluorinated methacrylates and the like and mixtures thereof. In another embodiment, the acrylate ester and/or methacrylate ester-containing monomers are hydrophobic monomers.

Representative examples of acrylate ester-containing monomers for use herein include, but are not limited to, methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and the like and mixtures thereof.

Representative examples of methacrylate ester-containing monomers for use herein include, but are not limited to, methyl methacrylate, ethyl methacrylate, propyl methacrylate, isopropyl methacrylate, n-butyl methacrylate, iso-butyl methacrylate, t-butyl methacrylate, n-hexyl methacrylate, 2-ethylbutyl methacrylate, 2-ethylhexyl methacrylate, cyclopropyl methacrylate, cyclobutyl methacrylate, cyclohexyl methacrylate, benzyl methacrylate, 2-phenoxyethyl methacrylate, phenyl methacrylate, 2-phenylethyl methacrylate, 3-phenylpropyl methacrylate, 3-phenoxypropyl methacrylate, 4-phenylbutyl methacrylate, 4-phenoxybutyl methacrylate, 4-methylphenyl methacrylate, 4-methylbenzyl methacrylate, 2-2-methylphenylethyl methacrylate, 2-3-methylphenylethyl methacrylate, 2-4-methylphenylethyl methacrylate and the like and mixtures thereof.

Suitable acrylamido-containing monomers may be represented by the general formulae II and III 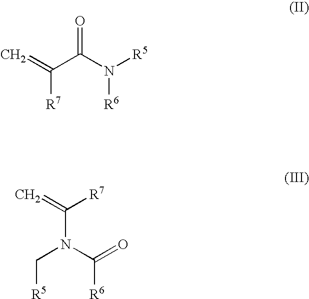
wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, or C₅-C₃₀ arylalkyl, substituted or unsubstituted, linear or branched, as defined above or R⁵ and R⁶ together with the nitrogen atom to which they are bonded are joined together to form a heterocyclic group and R⁷ is H or CH₃.

Representative examples of acrylamido-containing monomers include, but are not limited to, acrylamide, N-methylacrylamide, N-ethylacrylamide, N-propylacrylamide, N-isopropylacrylamide, N-butylacrylamide, N,N-dimethylacrylamide, N,N-d iethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide, N-vinylpyrrolidone and the like and mixtures thereof. In one embodiment, the acrylamido-containing monomers are hydrophilic monomers.

Generally, the monomeric mixture can contain from about 10% w/w to about 80% w/w and preferably from about 20% w/w to about 50% w/w of the acrylate ester and/or methacrylate ester-containing monomer(s) and from about 90% w/w to about 10% w/w and preferably from about 80% w/w to about 30% w/w of the acrylamido-containing monomer(s).

If desired, the monomeric mixture for use in forming the drug delivery systems of the present invention can further contain one or more crosslinking agents. Preferably, the crosslinking agent is one that is copolymerized with the reactive monomers. Suitable crosslinking agents include, but are not limited to, any di- or multi-functional crosslinking agent and the like and mixtures thereof. Representative examples of such crosslinkers include, but are not limited to, tripropylene glycerol diacrylate, ethylene glycol dimethacrylate, tetraethylene glycol dimethacrylate, poly(ethylene glycol diacrylate) (PEG400 or PEG600), methylene bis acrylamide and the like and mixtures thereof. If used, the crosslinking agent is used in an effective amount, by which is meant an amount that is sufficient to cause crosslinking of the monomeric mixture and polymerization with the one or more pharmaceutically active agents to produce the desired drug delivery system. The amount of the crosslinking agent will ordinarily range from about 0.05% w/w to about 20% w/w and preferably from about 0.1% w/w to about 10% w/w.

Generally, pharmaceutically active agents or drugs useful in the matrix controlled diffusion drug delivery systems of the present invention can be any compound, composition of matter, or mixtures thereof that can be delivered from the drug delivery system to produce a beneficial and useful result to, for example, the eye, especially an agent effective in obtaining a desired local or systemic physiological or pharmacological effect. Examples of such agents include, but are not limited to, anesthetics and pain killing agents such as lidocaine and related compounds, benzodiazepam and related compounds and the like; anti-cancer agents such as 5-fluorouracil, adriamycin and related compounds and the like; anti-fungal agents such as fluconazole and related compounds and the like; anti-viral agents such as trisodium phosphomonoformate, trifluorothymidine, acyclovir, ganciclovir, DDI, AZT and the like; cell transport/mobility impending agents such as colchicine, vincristine, cytochalasin B and related compounds and the like; antiglaucoma drugs such as beta-blockers, e.g., timolol, betaxolol, atenalol, and the like; antihypertensives; decongestants such as phenylephrine, naphazoline, tetrahydrazoline and the like; immunological response modifiers such as muramyl dipeptide and related compounds and the like; peptides and proteins such as cyclosporin, insulin, growth hormones, insulin related growth factor, heat shock proteins and related compounds and the like; steroidal compounds such as dexamethasone, prednisolone and related compounds and the like; low solubility steroids such as fluocinolone acetonide and related compounds and the like; carbonic anhydrase inhibitors; diagnostic agents; antiapoptosis agents; gene therapy agents; sequestering agents; reductants such as glutathione and the like; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen and the like; nutrients and vitamins: enzyme inhibitors: antioxidants; anticataract drugs; aldose reductase inhibitors; cytoprotectants; cytokines, cytokine inhibitors, and cytokin protectants; uv blockers; mast cell stabilizers; anti neovascular agents such as antiangiogenic agents, e.g., matrix metalloprotease inhibitors and the like.

Representative examples of additional pharmaceutically active agent for use herein include, but are not limited to, neuroprotectants such as nimodipine and related compounds and the like; antibiotics such as tetracycline, chlortetracycline, bacitracin, neomycin, polymyxin, gramicidin, oxytetracycline, chloramphenicol, gentamycin, erythromycin and the like; anti-infectives; antibacterials such as sulfonamides, sulfacetamide, sulfamethizole, sulfisoxazole; nitrofurazone, sodium propionate and the like; antiallergenics such as antazoline, methapyriline, chlorpheniramine, pyrilamine, prophenpyridamine and the like; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triminolone and the like; miotics; anti-cholinesterase such as pilocarpine, eserine salicylate, carbachol, di-isopropyl fluorophosphate, phospholine iodine, demecarium bromide and the like; miotic agents; mydriatics such as atropine sulfate, cyclopentolate, homatropine, scopolamine, tropicamide, eucatropine, hydroxyamphetamine and the like; svmpathomimetics such as epinephrine and the like; and prodrugs such as, for example, those described in Design of Prodrugs, edited by Hans Bundgaard, Elsevier Scientific Publishing Co., Amsterdam, 1985. In addition to the foregoing agents, other agents suitable for treating, managing, or diagnosing conditions in a mammalian organism may be entrapped in the copolymer and administered using the drug delivery systems of the current invention. Once again, reference may be made to any standard pharmaceutical textbook such as, for example, Remington's Pharmaceutical Sciences for pharmaceutically active agents.

Any pharmaceutically acceptable form of the foregoing pharmaceutically active agent may be employed in the practice of the present invention, e.g., the free base; free acid; pharmaceutically acceptable salts, esters or amides thereof, e.g., acid additions salts such as the hydrochloride, hydrobromide, sulfate, bisulfate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulfate salts and the like; alkali or alkaline earth metal salts such as the sodium, calcium, potassium and magnesium salts and the like; hydrates; enantiomers; isomers; stereoisomers; diastereoisomers; tautomers; polymorphs, mixtures thereof, prodrugs thereof or racemates or racemic mixtures thereof.

Actual dosage levels of the pharmaceutically active agent(s) in the drug delivery systems of the present invention may be varied to obtain an amount of the pharmaceutically active agent(s) that is effective to obtain a desired therapeutic response for a particular system and method of administration. The selected dosage level therefore depends upon such factors as, for example, the desired therapeutic effect, the route of administration, the desired duration of treatment, and other factors. The total daily dose of the pharmaceutically active agent(s) administered to a host in single or divided doses can vary widely depending upon a variety of factors including, for example, the body weight, general health, sex, diet, time and route of administration, rates of absorption and excretion, combination with other drugs, the severity of the particular condition being treated, etc. Generally, the amounts of pharmaceutically active agent(s) present in the drug delivery systems of the present invention can range from about 1% w/w to about 60% w/w and preferably from about 5% w/w to about 50% w/w.

The drug delivery systems of the present invention can be prepared by polymerizing the monomeric mixture containing at least the one or more acrylate ester and/or methacrylate ester-containing monomers, one or more acrylamido-containing monomers and optional crosslinking agent(s) with a therapeutically effective amount of one or more suitable pharmaceutically active agents. The resulting drug delivery systems can be copolymers, in random or block sequences, containing one or more acrylate ester and/or methacrylate ester-containing units and one or more acrylamido-containing units optionally crosslinked with the one or more crosslinking agents and polymerized with the suitable pharmaceutically active agent(s) such that the pharmaceutically active agent(s) is covalently bound to the copolymer. As one skilled in the art will readily appreciate, the resulting polymerization product can contain some free pharmaceutically active agent(s) and starting monomer(s) which are not covalently bound. If desired, these reactants can be removed from the resulting product by conventional techniques.

In general, the polymerization reaction can be conducted neat, that is, the acrylate ester and/or methacrylate ester-containing monomer(s), acrylamido-containing monomer(s), optional crosslinking agent(s) and the pharmaceutically active agent(s) are combined in the desired ratio, and then exposed to, for example, ultraviolet (UV) light, visible light or electron beams in the presence of one or more photoinitiator(s) or at a suitable temperature, for a time period sufficient to form the drug delivery system. Suitable reaction times will ordinarily range from about 1 minute to about 24 hours and preferably from about 1 hour to about 4 hours.

The use of UV or visible light in combination with photoinitiators is well known in the art and is particularly suitable for formation of the resulting polymerization product. Numerous photoinitiators of the type in question here are commercial products. Photoinitiators enhance the rapidity of the curing process when the photocurable compositions as a whole are exposed to, for example, ultraviolet radiation. Suitable photoinitiators which are useful for polymerizing the polymerizable mixture of monomers can be commercially available photoinitiators. They are generally compounds which are capable of initiating the radical reaction of olefinically unsaturated double bonds on exposure to light with a wavelength of, for example, about 260 to about 480 nm.

Examples of suitable photoinitiators for use herein include, but are not limited to, one or more photoinitiators commercially available under the “IRGACURE”, “DAROCUR” and “SPEEDCURE” trade names (manufactures by Ciba Specialty Chemicals, also obtainable under a different name from BASF, Fratelli Lamberti and Kawaguchi), e.g., “IRGACURE” 184 (1-hydroxycyclohexyl phenyl ketone), 907 (2-methyl-1-[4-(methylthio)phenyl]-2-morpholino propan-1-one), 369 (2-benzyl-2-N,N-dimethylamino-1-(4-morpholinophenyl)-1-butanone), 500 (the combination of 1-hydroxy cyclohexyl phenyl ketone and benzophenone), 651 (2,2-dimethoxy-2-phenyl acetophenone), 1700 (the combination of bis(2,6-dimethoxybenzoyl-2,4,4-trimethyl pentyl)phosphine oxide and 2-hydroxy-2-methyl-1-phenyl-propan-1-one), and 819 [bis(2,4,6-trimethyl benzoyl)phenyl phosphine oxide] and “DAROCUR” 1173 (2-hydroxy-2-methyl-1-phenyl-1-propan-1-one) and 4265 (the combination of 2,4,6-trimethylbenzoyldiphenyl-phosphine oxide and 2-hydroxy-2-methyl-1-phenyl-propan-1-one); and the like and mixtures thereof. Other suitable photoinitiators for use herein include, but are not limited to, alkyl pyruvates, such as methyl, ethyl, propyl, and butyl pyruvates, and aryl pyruvates, such as phenyl, benzyl, and appropriately substituted derivatives thereof. Generally, the amount of photoinitiator can range from about 0.05% w/w to about 5% w/w and preferably from about 0.1% w/w to about 1% w/w.

The polymerization of the monomeric mixture containing at least the acrylate ester and/or methacrylate ester-containing monomer(s), acrylamido-containing monomer(s), optional crosslinking agent(s) with the pharmaceutically active agent(s) can be carried out in any known manner. The important factors are intimate contact of the reactive monomers and suitable pharmaceutically active agent(s) and then exposing the reactive monomers and suitable pharmaceutically active agent(s) to, for example, ultraviolet (UV) light, visible light or electron beams in the presence of one or more photoinitiator(s) or at a suitable temperature, for a time period sufficient to form the polymerization product. It may be necessary, depending on the particular pharmaceutically active agent used, to convert the pharmaceutically active agent to a polymerized monomer by attaching a polymerizable functional group on the active agent by methods well known in the art prior to adding it to the reaction mixture. The components in the reaction mixture can also be added continuously to a stirred reactor or can take place in a tubular reactor in which the components can be added at one or more points along the tube.

The matrix controlled diffusion drug delivery systems of the present invention may be manufactured in any suitable form, shape, e.g., circular, rectangular, tubular, square and triangular shapes, or size suitable for the treatment which they are intended to be used. Methods of forming the subject matrix controlled diffusion drug delivery systems include, but are not limited to, cast molding, injection/compression molding, extrusion, and other methods known to those skilled in the art. For example, the drug delivery system may be sized and configured for back of the eye delivery such as an inner back of the eye implant, e.g., a hollow cylinder or tube having a first cross dimension (diameter, width), ranging from about 0.025 mm to about 10 mm and a second cross dimension, such as length, from about 0.2 mm to about 10 mm.

Alternatively, the drug delivery system can be in the form of a solution, suspension, solution/suspension, microsphere or nanosphere using a pharmaceutically acceptable carrier well known in the art. Additionally, the solution, suspension, solution/suspension, microsphere or nanosphere can contain one or more pharmaceutically acceptable excipients such as suspending agents, e.g., sodium carboxymethyl cellulose, methylcellulose, hydroxypropylmethylcellulose, sodium alginate, poly(N-vinylpyrrolidone), gum tragacanth and gum acacia; dispersing or wetting agents, e.g., naturally occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. Once manufactured, the subject matrix controlled diffusion drug delivery systems are packaged and sterilized using customary methods known to those skilled in the art.

Matrix controlled diffusion drug delivery systems of the present invention may be used in a broad range of therapeutic applications. The matrix controlled diffusion drug delivery systems of the present invention are particularly useful in the treatment of an ophthalmic state, disease, disorder, injury or condition. Representative examples of such an ophthalmic state, disease, disorder, injury or condition include, but is not limited to, diabetic retinopathy, glaucoma, macular degeneration, retinitis pigmentosa, retinal tears or holes, retinal-detachment, retinal ischemia, acute retinopathies associated with trauma, inflammatory mediated degeneration, post-surgical complications, damage associated with laser therapy including photodynamic therapy (PDT), surgical light induced iatrogenic retinopathy, drug-induced retinopathies, autosomal dominant optic atrophy, toxic/nutritional amblyopias; leber's hereditary optic neuropathy (LHOP), other mitochondrial diseases with ophthalmic manifestations or complications, angiogenesis; atypical RP; bardet-biedl syndrome; blue-cone monochromacy; cataracts; central areolar choroidal dystrophy; choroideremia; cone dystrophy; rod dystrophy; cone-rod dystrophy; rod-cone dystrophy; congenital stationary night blindness; cytomegalovirus retinitis; diabetic macular edema; dominant drusen; giant cell arteritis (GCA); goldmann-favre dystrophy; graves' opthalmopathy; gyrate atrophy; hydroxychloroquine; iritis; juvenile retinoschisis; keams-sayre syndrome; lawrence-moon bardet-biedl syndrome; leber congenital amaurosis; lupus-induced cotton wool spots; macular degeneration, dry form; macular degeneration, wet form; macular drusen; macular dystrophy; malattia leventinese; ocular histoplasmosis syndrome; oguchi disease; oxidative damage; proliferative vitreoretinopathy; refsum disease; retinitis punctata albescens; retinopathy of prematurity; rod monochromatism; RP and usher syndrome; scleritis; sector RP; sjogren-larsson syndrome; sorsby fundus dystrophy; stargardt disease and other retinal diseases.

The matrix controlled diffusion drug delivery systems of the present invention can be administered to a mammal in need of treatment by way of a variety of routes. For example, the drug delivery system may be used by implantation within a portion of the body in need of localized drug delivery, e.g., the interior portion of an eye. However, the subject matrix controlled diffusion drug delivery system may likewise be used in accordance with other surgical procedures known to those skilled in the field of opthalmology. For example, the drug delivery systems can be administered to the region of the eye in need of treatment employing instruments known in the art, e.g., a flexible microcatheter system or cannula disclosed in U.S. Patent Application Publication No. 2002/0002362, or the intraretinal delivery and withdrawal systems disclosed in U.S. Pat. Nos. 5,273,530 and 5,409,457, the contents of each which are incorporated by reference herein. The pharmaceutically active agent may be released from the drug delivery device over a sustained and extended period of time. Optionally, the drug release rate may also be controlled through the attachment of an inert diffusion barrier by way of, for example, surface treatment of the drug delivery device. The surface treatment may be applied through a variety of surface treatment techniques known in the art, e.g., oxidative plasma, evaporative deposition, dip coating or extrusion techniques.

The following examples are provided to enable one skilled in the art to practice the invention and are merely illustrative of the invention. The examples should not be read as limiting the scope of the invention as defined in the claims.

### EXAMPLE 1

To 70 parts of N,N-dimethylacrylamide (DMA) was added 30 parts of methylmethacrylate (MMA), 3 parts of ethylene glycol dimethacrylate (as a crosslinking agent), and 1.0% Irgacure 819 (as a photoinitiator). To this reaction mixture was added 20% w/w of fluocinolone acetonide (FA). The solution was added to Teflon tubes (0.5 mm in diameter) available from Boramed (Durham, N.C.) and polymerized using visible light polymerization techniques. The cure conditions consisted of two hours of visible light irradiation. Following the cure, the drug loaded copolymer was removed from the tube resulting in a release device having dimensions of 5 mm by 0.5 mm.

### EXAMPLE 2

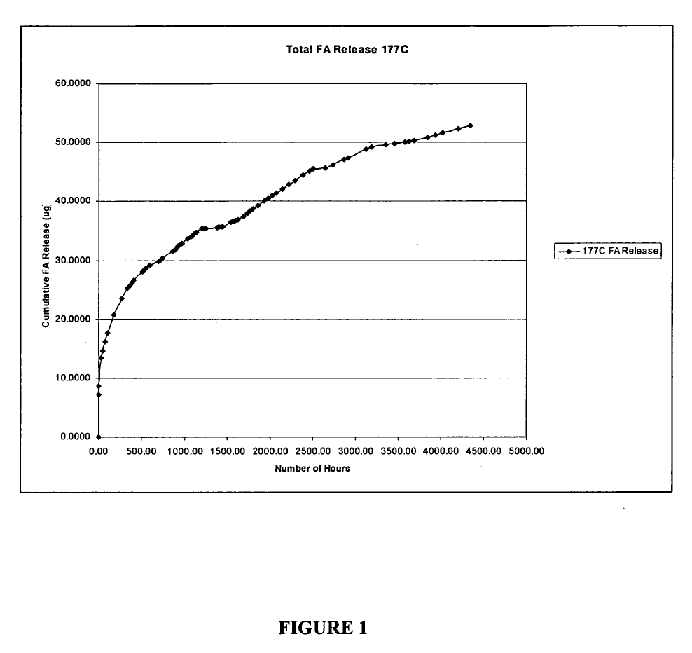

The sample as prepared in Example 1 was placed in 3 cc of borate buffer in a sealed glass tube and the amount of FA release was monitored at 34° C. At periodic intervals, 3 cc of solution was removed and replaced with 3 cc of fresh borate. The solution was analyzed by liquid chromatography for FA. The release rate per day and percent cumulative release were determined as illustrated in FIG. 1. A zero-order drug release was obtained shortly after the initial burst (for sample 177° C.).

### EXAMPLE 3

To 30 parts of DMA was added 70 parts of MMA, 3 parts of ethylene glycol dimethacrylate (as a crosslinking agent), and 1.0% Irgacure 819 (as a photoinitiator). To this reaction mixture was added 40% w/w of FA. The solution was added to Teflon tubes (0.5 mm in diameter) available from Boramed (Durham, N.C.) and polymerized using visible light polymerization techniques. The cure conditions consisted of two hours of visible light irradiation. Following the cure, the drug loaded copolymer was removed from the tube resulting in a release device having dimensions of 5 mm by 0.5 mm.

### EXAMPLE 4

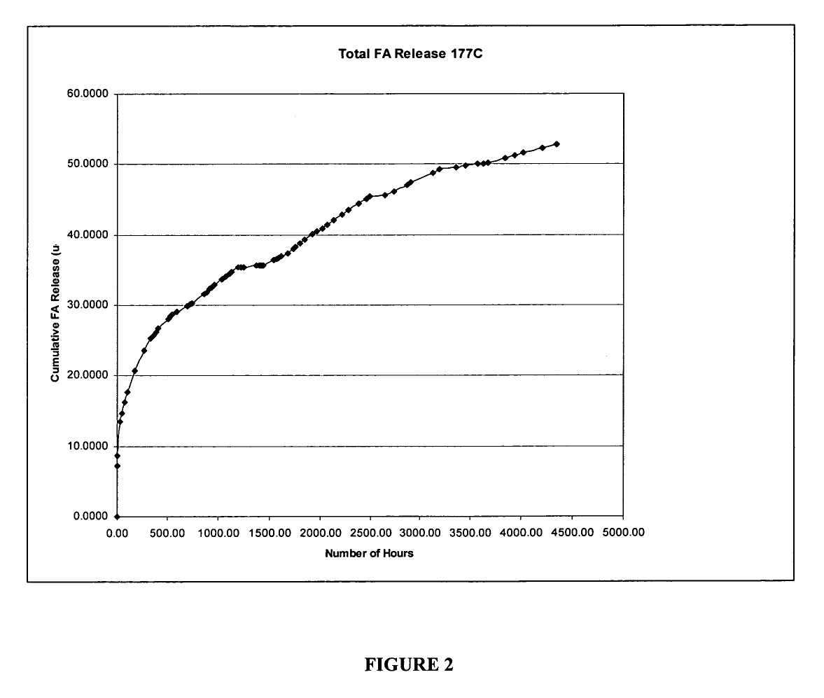

The sample as prepared in Example 3 was placed in 3 cc of borate buffer in a sealed glass tube and the amount of FA release was monitored at 34° C. At periodic intervals, 3 cc of solution was removed and replaced with 3 cc of fresh borate. The solution was analyzed by liquid chromatography for FA. The release rate per day and percent cumulative release were determined as illustrated in FIG. 2. A zero-order drug release was obtained shortly after the initial burst (for sample 177° C.).

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A matrix controlled diffusion drug delivery system comprising a therapeutically effective amount of one or more pharmaceutically active agents polymerized with a monomeric mixture comprising one or more acrylate ester and/or methacrylate ester-containing monomers and one or more acrylamido-containing monomers.

2. The matrix controlled diffusion drug delivery system of claim 1, wherein the acrylate ester and/or methacrylate ester-containing monomer is represented by the general formula I: 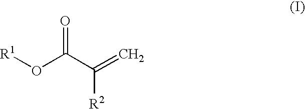
wherein R¹ is a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, an ether or polyether containing group, substituted or unsubstituted, linear or branched, and R² is H or CH₃.

3. The matrix controlled diffusion drug delivery system of claim 1, wherein the acrylate ester and/or methacrylate ester-containing monomer is selected from the group consisting of a methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and mixtures thereof.

4. The matrix controlled diffusion drug delivery system of claim 1, wherein the acrylamido-containing monomer is represented by the general formulae II and III: 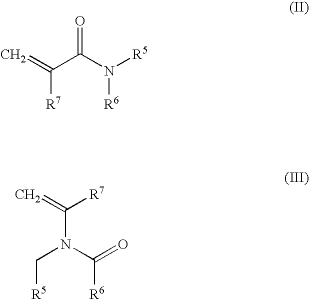
wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, or C₅-C₃₀ arylalkyl, substituted or unsubstituted, linear or branched, or R⁵ and R⁶ together with the nitrogen atom to which they are bonded are joined together to form a heterocyclic group and R⁷ is H or CH₃.

5. The matrix controlled diffusion drug delivery system of claim 1, wherein the acrylamido-containing monomer is selected from the group consisting of acrylamide, N-methylacrylamide, N-ethylacrylamide, N-propylacrylamide, N-isopropylacrylamide, N-butylacrylamide, N,N-dimethylacrylamide, N,N-diethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide and mixtures thereof.

6. The matrix controlled diffusion drug delivery system of claim 1, wherein the acrylate ester and/or methacrylate ester-containing monomer is a hydrophobic acrylate ester and/or methacrylate ester-containing monomer and the acrylamido-containing monomer is a hydrophilic acrylamido-containing monomer.

7. The matrix controlled diffusion drug delivery system of claim 1, wherein the acrylate ester and/or methacrylate ester-containing monomer is present in the monomeric mixture in an amount of about 10% w/w to about 80% w/w and the acrylamido-containing monomer is present in the monomeric mixture in an amount of from about 90% w/w to about 10% w/w.

8. The matrix controlled diffusion drug delivery system of claim 1, wherein the acrylate ester and/or methacrylate ester-containing monomer is present in the monomeric mixture in an amount of from about 20% w/w to about 50% w/w and the acrylamido-containing monomer is present in the monomeric mixture in an amount of from about 80% w/w to about 30% w/w.

9. The matrix controlled diffusion drug delivery system of claim 1, wherein the monomeric mixture further comprises one or more crosslinking agents.

10. The matrix controlled diffusion drug delivery system of claim 9, wherein the crosslinking agent is selected from the group consisting of tripropylene glycerol diacrylate, ethylene glycol dimethacrylate, tetraethylene glycol dimethacrylate, poly(ethylene glycol diacrylate), methylene bis acrylamide and mixtures thereof.

11. The matrix controlled diffusion drug delivery system of claim 1, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

12. The matrix controlled diffusion drug delivery system of claim 1, wherein the one or more pharmaceutically active agents is selected from the group consisting of an anticholinergic, anticoagulant, antifibrinolytic, antihistamine, antimalarial, antitoxin, chelating agent, hormone, immunosuppressive, thrombolytic, vitamin, protein, salt, desensitizer, prostaglandin, amino acid, metabolite, antiallergenic and mixtures thereof.

13. The matrix controlled diffusion drug delivery system of claim 1, which is sized and configured for back of the eye delivery.

14. The matrix controlled diffusion drug delivery system of claim 10, which is sized and configured for back of the eye delivery.

15. The matrix controlled diffusion drug delivery system of claim 1, in a form of a solution, suspension, solution/suspension, microsphere or nanosphere.

16. The matrix controlled diffusion drug delivery system of claim 1, in a form of a semi-solid or solid article suitable for ocular implant.

17. A matrix controlled diffusion drug delivery system comprising a therapeutically effective amount of one or more pharmaceutically active agents polymerized with a copolymer obtained from the copolymerization of one or more acrylate ester and/or methacrylate ester-containing monomers with one or more acrylamido-containing monomers.

18. The matrix controlled diffusion drug delivery system of claim 17, wherein the acrylate ester and/or methacrylate ester-containing monomer is represented by the general formula I: 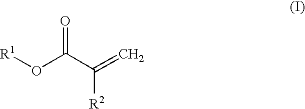
wherein R¹ is a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, an ether or polyether containing group, substituted or unsubstituted, linear or branched, and R² is H or CH₃.

19. The matrix controlled diffusion drug delivery system of claim 17, wherein the acrylate ester and/or methacrylate ester-containing monomer is selected from the group consisting of a methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and mixtures thereof and the acrylamido-containing monomer is selected from the group consisting of acrylamide, N-methylacrylamide, N-ethylacrylamide, N-propylacrylamide, N-isopropylacrylamide, N-butylacrylamide, N,N-dimethylacrylamide, N,N-diethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide and mixtures thereof.

20. The matrix controlled diffusion drug delivery system of claim 17, wherein the acrylamido-containing monomer is represented by the general formulae II and III: 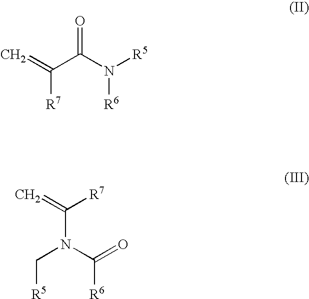
wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, or C₅-C₃₀ arylalkyl, substituted or unsubstituted, linear or branched, or R⁵ and R⁶ together with the nitrogen atom to which they are bonded are joined together to form a heterocyclic group and R⁷ is H or CH₃.

21. The matrix controlled diffusion drug delivery system of claim 17, wherein the acrylate ester and/or methacrylate ester-containing monomer is a hydrophobic acrylate ester and/or methacrylate ester-containing monomer and the acrylamido-containing monomer is a hydrophilic acrylamido-containing monomer.

22. The matrix controlled diffusion drug delivery system of claim 17, wherein the copolymerization further comprises one or more crosslinking agents.

23. The matrix controlled diffusion drug delivery system of claim 22, wherein the crosslinking agent is selected from the group consisting of tripropylene glycerol diacrylate, ethylene glycol dimethacrylate, tetraethylene glycol dimethacrylate, poly(ethylene glycol diacrylate), methylene bis acrylamide and mixtures thereof.

24. The matrix controlled diffusion drug delivery system of claim 17, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

25. The matrix controlled diffusion drug delivery system of claim 1, wherein the one or more pharmaceutically active agents is selected from the group consisting of an anticholinergic, anticoagulant, antifibrinolytic, antihistamine, antimalarial, antitoxin, chelating agent, hormone, immunosuppressive, thrombolytic, vitamin, protein, salt, desensitizer, prostaglandin, amino acid, metabolite, antiallergenic and mixtures thereof.

26. The matrix controlled diffusion drug delivery system of claim 17, which is sized and configured for back of the eye delivery.

27. The matrix controlled diffusion drug delivery system of claim 17, in a form of a solution, suspension, solution/suspension, microsphere or nanosphere.

28. The matrix controlled diffusion drug delivery system of claim 17, in a form of a semi-solid or solid article suitable for ocular implant.

29. A process for preparing a matrix controlled diffusion drug delivery system, the process comprising polymerizing a therapeutically effective amount of one or more pharmaceutically active agents with a monomeric mixture comprising one or more acrylate ester and/or methacrylate ester-containing monomers and one or more acrylamido-containing monomers.

30. The process of claim 29, wherein the acrylate ester and/or methacrylate ester-containing monomer is represented by the general formula I: 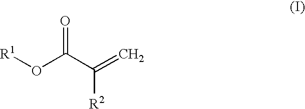
wherein R¹ is a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, an ether or polyether containing group, substituted or unsubstituted, linear or branched, and R² is H or CH₃.

31. The process of claim 29, wherein the acrylamido-containing monomer is represented by the general formulae II and III: 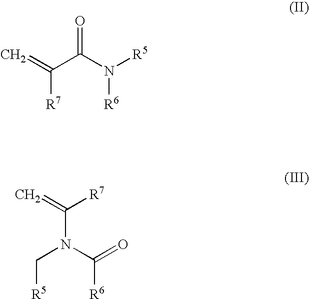
wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, or C₅-C₃₀ arylalkyl, substituted or unsubstituted, linear or branched, or R⁵ and R⁶ together with the nitrogen atom to which they are bonded are joined together to form a heterocyclic group and R⁷ is H or CH₃.

32. The process of claim 29, wherein the acrylate ester and/or methacrylate ester-containing monomer is a hydrophobic acrylate ester and/or methacrylate ester-containing monomer and the acrylamido-containing monomer is a hydrophilic acrylamide-containing monomer.

33. The process of claim 29, wherein the monomeric mixture further comprises one or more crosslinking agents.

34. The process of claim 29, wherein the step of polymerizing comprises exposing the monomeric mixture and one or more pharmaceutically active agents to ultraviolet or visible light in the presence of one or more photoinitiators for a time sufficient to form a polymerization product.

35. A process for preparing a matrix controlled diffusion drug delivery system, the process comprising polymerizing a therapeutically effective amount of one or more pharmaceutically active agents with a copolymer obtained from the copolymerization of one or more acrylate ester and/or methacrylate ester-containing monomers with one or more acrylamido-containing monomers.

36. A matrix controlled diffusion drug delivery system comprising a therapeutically effective amount of one or more pharmaceutically active agents polymerized with a copolymer comprising one or more acrylate ester and/or methacrylate ester-containing units and one or more acrylamido-containing units.

37. A method of treating an ophthalmic state, disease, disorder, injury or condition, the method comprising administering to a mammal in need of such treatment the matrix controlled diffusion drug delivery system of claim 1.

38. The method of claim 37, wherein the step of administering comprises:
creating an incision within an eye; and
implanting the matrix controlled diffusion drug delivery system within the eye through the incision.

39. The method of claim 37, wherein the step of administering comprises:
injecting the matrix controlled diffusion drug delivery system within an eye.

40. A method of treating an ophthalmic state, disease, disorder, injury or condition, the method comprising administering to a mammal in need of such treatment the matrix controlled diffusion drug delivery system of claim 13.

41. The method of claim 40, wherein the step of administering comprises:
creating an incision within an eye; and
implanting the matrix controlled diffusion drug delivery system within the eye through the incision.

42. The method of claim 40, wherein the step of administering comprises:
injecting the matrix controlled diffusion drug delivery system within an eye.
